(19) United States
(12) Patent Application Publication
(10) Pub. No.: US 2023/0293035 A1
(43) Pub. Date: Sep. 21, 2023

(54) Cardiac Cycle Analysis Method and System

(71) Applicant: Vital Metrix Inc., Huntsville, AL (US)

(72) Inventors: Jason Heym, Huntsville, AL (US); Sami Bayyuk, Huntsville, AL (US); Alton Reich, Huntsville, AL (US)

(21) Appl. No.: 17/909,523

(22) PCT Filed: Mar. 7, 2021
(86) PCT No.: PCT/US2021/021280

§ 371 Date: Sep. 6, 2022

Related U.S. Application Data
(60) Provisional application No. 62/986,681, filed on Mar. 7, 2020.

(51) Int. Cl. A61B 5/029; A61B 5/021; A61B 5/02

(52) U.S. Cl. CPC A61B 5/029; A61B 5/02108; A61B 5/02007; A61B 5/1455

(57) ABSTRACT

A method and system for processing pulse plethysmography detects peaks, troughs, and amplitudes for a series of measured cycles and identifies measured cycles that do not meet selected quality criteria. Cycles that do not meet selected quality criteria are removed and the remaining cycles are stitched together. The system and method are useful for improving the quality and reliability of cardiovascular parameters such as stroke volume and cardiac output calculated using plethysmography data as input.

### BACKGROUND OF THE INVENTION

### Field of the Invention

The present invention relates to devices, methods, and systems that measure cardiac output, stroke volume, and/or arterial pressure using data obtained by pulse plethysmography methods including photoplethysmography.

### Description of Related Art

Plethysmography involves the measurement of changes in volume within all or a part of the body. Changes in the volume of various parts of the body can be measured in different ways to detect or monitor a variety of medical conditions by measuring a variety of physiological parameters. For example, air cuff plethysmography (CPG) measures changes in the circumference of a limb or digit by recording the changes in pressure in an air-filled cuff surrounding the limb or digit. Based upon changes in volume, CPG is used to measure blood flow and provides useful information related to chronic venous diseases. Impedance plethysmography, also called dielectrography, involves the measurement of changes in electrical impedance between electrodes placed on opposite sides of a part of the body to indirectly measure changes in volume in the path of current flowing between the electrodes.

Inductance plethysmography is a method in which an alternating current is applied through a loop of wire worn around the chest or abdomen that generates a magnetic field normal to the orientation of the loop. Changes in the area enclosed by the loop create opposing currents within the loop directly proportional to the rate of change in the area. Unlike Inductance plethysmography, no electrical current passes through the body. Respiratory inductance plethysmography is used to calculate chest and abdominal expansion, respiratory rate, respiratory pattern, and tidal volume. Electromagnetic inductance plethysmography uses a vest comprising conductive elements that encircle the torso to quantify chest and abdominal volume changes as the conductive elements move in a magnetic field.

Photoplethysmography (PPG) measures changes in blood volume within a tissue caused by the pulse of blood pressure (BP) through the vasculature in the tissue. The blood volume change is detected by measuring the amount of light transmitted or reflected to a sensor from a light source used to illuminate the skin. Transmission photoplethysmographs (PPGs) can also be obtained using endoscopic methods. The shape of the PPG waveform differs from subject to subject, and varies with the location and manner in which the pulse oximeter is contacted with or attached to the body.

Recently, methods and apparatus for non-invasively measuring left ventricular stroke volume (SV), cardiac output (CO), and other physiological parameters have been developed that involve a type of physiological sensor data processing that makes these measurements possible. U.S. Pat. No. 8,494,829 discloses a method for processing hemodynamic and electrodynamic sensor data to calculate SV. The hemodynamic and electrodynamic sensor data can be obtained noninvasively and is processed using a model representing hemodynamic and electrodynamic physiology and repetitively fusing data from two sensors. U.S. Pat. No. 8,494,829 is incorporated herein by reference in its entirety, in particular the description of the physical model in columns 5-20. U.S. Pat. No. 9,649,036 describes a method for analyzing PPG and electrodynamic signals using a digital signal processor with a dynamic state-space model (DSSM) having a process model relating the PPG and electrodynamic signals originating in the body to systolic contraction. U.S. Pat. No. 9,649,036 is incorporated herein by reference, in particular the example of a pulse oximeter (PO) with probabilistic data filtering in columns 15-19.

U.S. Pat. No. 9,060,722 describes a method for estimating SV using sensor data from a PPG using a dual estimation algorithm with a DSSM that mathematically represents physiological processes that produce SV and the data collected by the PPG. U.S. Pat. No. 9,060,722 is incorporated herein by reference, in particular the description of a dual estimation algorithm and DSSM in columns 6-14. U.S. Pat. No. 9,375,171 describes a method for estimating SV from BP and PPG data using a dual estimation DSSM with a hemodynamic process model.

WO 2010/0274102 A1 discloses a PO system and a processing method that measures SV and CO. The PO system comprises a data processor configured to perform a method that combines a probabilistic processor and a physiological model of the cardiovascular system in a DSSM that can remove contaminating noise and artifacts from the PO sensor output and measure blood oxygen saturation, heart rate (HR), SV, aortic pressure and systemic pressures. The DSSM comprises a mathematical model of the cardiovascular system which models physiological processes that produce the pulses measured by the PO. One of the models comprises variables and parameters including aortic pressure, radial pressure, peripheral resistance, aortic impedance, and blood density. The model uses data from the PO, which includes measured HR.

EP 2512325 describes a method for processing PO data using a dynamic state space model to extract an estimated value for one or more of total blood volume, SV, vasomotor tone, and autonomous nervous system tone. A microprocessor receives data into a dynamic state space model that mathematically represents physiological processes that produce physiological parameters measured by the PO.

While the above cited inventions can provide accurate and medically useful measurements of cardiovascular parameters including CO and SV, there are circumstances under which measured plethysmographic waveforms have characteristics that do not permit the measurement SV and CO or result in measurements of SV and CO with lower than desirable accuracy. These measurements of SV, CO, and other cardiovascular parameters involve the use of plethysmographic waveform (PW) data as input into a computer model of the cardiovascular system.

The quality of the PW data used as input has an effect on the quality of the output, which includes measured SV, CO, and/or other cardiovascular parameters, depending on the cardiovascular system model used. Motion of the plethysmographic sensor or the patient, including the movement of breathing, cause artifacts in the measured plethysomgraphic waveform. Beat to beat changes in HR and SV, body movement, and changes in peripheral resistance over time for an individual complicate the accurate measurement of SV and CO. For a population, a wide range of heart abnormalities, cardiovascular diseases, body mass indices, body sizes, ages and many other physiological parameters can also complicate the measurement of CO and SV because the PW data in a population can have a variety of shapes, making the analysis of the input data for inclusion as input into cardiovascular models difficult. The PW data measured for certain patients can be difficult or impossible to process using a cardiovascular system model because the PW shapes differ significantly from the average patient population.

In summary, a number of circumstances exist under which measured PWs include artifacts or have other characteristics that do not permit the measurement SV, CO, and other cardiovascular parameters at all, or result in measurements of SV and CO with lower than desirable accuracy.

### BRIEF SUMMARY OF THE INVENTION

Embodiments of the present invention preferably seek to mitigate, alleviate or eliminate one or more deficiencies, disadvantages or issues in the art, such as the above identified, singly or in any combination, by providing a system and method for processing PW data representing blood flow to improve the reliability and accuracy SV and CO measurements made using the PW data as input.

In one aspect, the present invention provides a computer implemented method for analyzing cardiac cycles in a set of PW data to produce input data for a method or processor that measures or calculates CO, SV, or other cardiovascular parameters using the processed data.

In another aspect, the present invention provides an apparatus or system for analyzing cardiac cycles in a set of PW data to produce input data for a processor that measures CO, SV, or other cardiovascular parameters using the processed data. The apparatus comprises a data processor comprising instructions for performing the method.

In yet another aspect, present invention provides a non-transitory computer-readable storage medium storing a program that causes a computer to execute a method for analyzing cardiac cycles in a set of PW data to produce input data for a processor that measures CO, SV, or other cardiovascular parameters using the processed data.

The method and system described improve the accuracy and reliability of cardiovascular parameter measurements made using pulse plethysmographic data by selecting portions of waveform data to be used for calculating cardiovascular parameters from the pulse plethysmographic data and selecting other portions of waveform data to be eliminated from use in calculating cardiovascular parameters.

### BRIEF DESCRIPTION OF THE DRAWINGS

The elements of the drawings are not necessarily to scale relative to each other, with emphasis placed instead upon clearly illustrating the principles of the disclosure. Like reference numerals designate corresponding parts throughout the several views of the drawings in which:

FIG. 1 is a flowchart of general processes that may be performed in an embodiment of a method for processing waveform data according to the invention;

FIG. 2a is a flowchart of steps that may be performed in a cycle data quality assessment process;

FIG. 2b is an example of PW data cycle data quality assessment by identification of major cycle features;

FIG. 3a is a flowchart of steps that may be performed in a coarse sieving process;

FIG. 3b is an example of PW data, approximated waveform, and residual error determined during coarse sieving;

FIG. 4a is a flowchart of steps that may be performed in a cardiac cycle analysis process;

FIG. 4b is an example of two skew-Gaussian functions used to approximate PW cycle data;

FIG. 4c is an example of PW cycles compared to approximated cycles using two skew-Gaussian functions;

FIG. 4d is an example of three skew-Gaussian functions used to approximate PW cycle data;

FIG. 4e is an example of PW cycles compared to approximated cycles using three skew-Gaussian functions;

FIG. 4f is an example of four skew-Gaussian functions used to approximate PW cycle data;

FIG. 4g is an example of PW cycles compared to approximated cycles using four skew-Gaussian functions; and

FIG. 5 is a flowchart indicating flow of data between components of an apparatus or system for analyzing cardiac cycles to measure CO, SV, or other cardiovascular parameters.

### DETAILED DESCRIPTION OF THE INVENTION

All art specific terms used herein are intended to have their art-accepted meanings in the context of the description unless otherwise indicated. All non art specific terms are intended to have their plain language meaning in the context of the description unless otherwise indicated. The term “parameter” in the context of a cardiovascular system includes physical factors such as stroke volume (SV), cardiac output (CO), aortic pressure, venous pressure, arterial pressure, flow rate, vessel inertance, vessel compliance, vessel resistance and the inertance, compliance, and resistance of collections of vessels or portions of the cardiovascular system. In the context of a computational model of a cardiovascular system, some of the physical factors may also be parameters in the modeling sense in that they may correspond to fixed input values into a computational model. Other physical factors, in the context of a computational model, correspond to variables for which values are calculated by a computational model. A cardiovascular system parameter may correspond to a variable of a computational model or may be calculated from one or more variables. Whether a cardiovascular system parameter corresponds to, or is calculated from, a variable and/or parameter of a computational model depends on the equations used in the computational model.

As used herein, plethysmography data is data collected from a human or nonhuman patient and wherein the data comprises a waveform representing pressure versus time or volume versus time for a blood flow in the patient.

Plethysmographic waveform data, or PW data, relates to measuring changes over time in blood volume, which can be converted to time varying changes in blood pressure (BP). Time varying pulse pressure may be measured directly or indirectly from plethysmography data. Plethysmography waveforms may comprise measured pulse volume vs. time or fraction of heart cycle. Plethysmography waveforms may comprise pulse pressure as measured by plethysmography vs. time or fraction of heart cycle.

The most commonly used plethysmography methods and apparatus are pulse oximetry or pulse oximeter (PO) devices that use absorption of light at two wavelengths corresponding to the absorption maxima for oxygenated and deoxygenated forms of hemoglobin. Changes in absorption are related to changes in blood volume and changes in pressure can be derived from changes in blood volume. Time varying blood volume may additionally or alternatively be measured using ultrasound to measure the diameter of blood vessels. Time varying pulse pressure waveforms may also be measured directly using a partially inflated blood pressure cuff, pressure transducers, strain gages or stretch sensors. The systems, apparatus, and methods are described herein using pressure as a parameter that changes with time. This is convenient because PO data is commonly converted to pressure vs. time data.

Pressure need not be used as the time varying variable of plethysmography data. Instead, PW cycles may be in the form of volume vs. time or absorption vs. time, for example. PO devices use two wavelengths so that amounts of oxygenated and deoxygenated hemoglobin may be compared to report a percent oxygenation of hemoglobin. While the usual PO data is used for the examples herein, the apparatus and methods herein may use data collected using, for example, only changes in absorption by oxygenated hemoglobin or changes in blood volume or BP derived therefrom. PO data is most often collected at fingertips but may additionally or optionally be measured on other extremities such as ears or toes and reflectance or scattering PO measurements may be made at locations on the body that are not compatible with transmission PO measurements.

In the following description, PO plethysmography data is used as an example of pulse plethysmography data for measuring SV and CO. Other types of plethysmography data may be in the form of, or converted to, pulsatile blood flow waveforms of pressure vs. time, volume vs. time, or light absorption vs. time. These can be used as well because volume, pressure, and absorption may be converted from one to the other depending on the method of data collection. PO data may come from a database of PO measurements made on subjects at a remote location and stored in a memory. The PO data may come directly from a PO while making measurements.

The plethysmography data processed as described may be used as input for data for measuring, calculating, or estimating SV, CO, and other cardiovascular system parameters, for example by methods using human cardiovascular system (HCS) models which may comprise a dynamic state-space model (DSSM). The data need not be human plethysmography data entered into computer implemented methods using cardiovascular system models of nonhuman animals to measure or calculate cardiovascular parameters. Examples of computer implemented methods for measuring cardiovascular parameters are described in U.S. Pat. Nos. 8,494,829; 9,649,036; 9,060,722; 9,375,171; WO 2010/0274102; and EP 2512325.

HR, SV, CO, and other cardiovascular parameters are not constant over time and may change from beat to beat. Any measurement of this type of parameter, therefore, is necessarily an averaged value. The constant variation in measured pulse pressures, duration of cycles, and shapes of PWs, are complicating factors for the measurement of SV, CO, and other cardiovascular parameters. In addition to these, movement of the subject and other physical disturbances cause artifacts in the data. A quality metric may applied to measured PW data to solve problems arising from artifacts and complicating factors in measured PW data by minimizing the effects of artifacts and outliers on subsequent processing steps. For example, an algorithm may select a set of individual cycles and/or runs of cycles that have, in combination, a high ratio of cycle amplitude to cycle amplitude variability, minimum localized plethysmography or pressure wave amplitude variability, and minimum HR variability.

The present invention provides a method for processing PW data to select cycles for use as input into a computer implemented method for measuring cardiovascular parameters using plethysmography pulse data. This method improves the accuracy of measured cardiovascular parameters and expands the application of computer implemented methods for measuring cardiovascular parameters to patients having PWs that are unusual in shape. This is accomplished by a combination of data quality assessment, data sieving, and curve fitting that select cycles from PW to be used as input for a computer implemented method for measuring cardiovascular parameters using plethysmography pulse data.

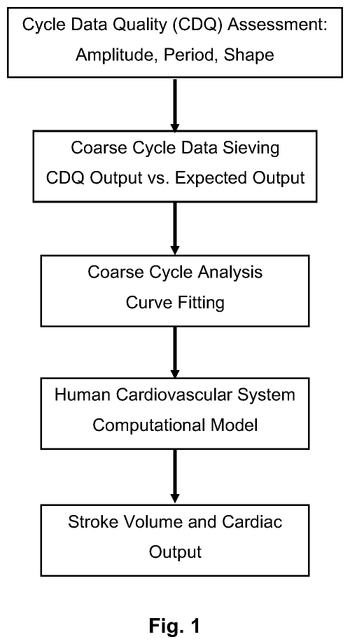

FIG. 1 provides an overview of the general processes that may be performed in an embodiment of a method for processing PW data. The data comprises a series of waveforms that corresponds to a series of pulses measured by a plethysmography measuring device, with each pulse arising from a heartbeat, or cycle of the heart. PW data, from a PO for example, is entered into a software module configured to perform a cycle data quality (CDQ) assessment algorithm that uses PW cycle features such as wave amplitude, wave shape, and cycle period to select or deselect each cycle for further processing. PW data for selected cycles are entered into a coarse cycle data sieving, or sieving, software module that compares the received CDQ data to an expected output for the CDQ data and coarsely approximates the shape of each cycle with a first mathematical formula. The algorithm compares the coarse approximation with the CDQ data and eliminates PW cycles based a cumulative error between the measured and approximated cycle waveforms. PW cycles that remain are entered into a software module that performs a cardiac cycle analysis (CCA) algorithm. The CCA module receives the coarsely sieved PW data and fits a second mathematical formula to each cycle waveform. The shape of the mathematical representation of the waveform for each cycle is compared to the shape of the corresponding measured waveform for that cycle. Cycles with measured and fitted waveforms that differ by a measured value above a threshold value are eliminated and the PW cycle data that passes the CCA may be made available as input data for a computer module that calculates a cardiovascular parameter, in this case SV and CO, using the CCA output data as input into a human cardiovascular system (HCS) model.

CDQ assessment, cycle data sieving, and cardiac cycle analysis serve as quality metric processes to identify measured cycles that do not meet selected quality criteria associated with these processes. The remaining measured cycles or mathematical representation of the remaining measured cycle waveforms are useful as input into computational models that calculate cardiovascular parameters from measured plethysmography waveform data. Depending on the computational model used, the cardiovascular parameters calculated may include SV, CO, aortic pressure, vascular inertance, compliance, resistance, and the like.

The sequence of processes shown in FIG. 1 is for one embodiment of a method for processing PW data. Other embodiments of such a method may comprise the same processes in a different order. For example, in one embodiment coarse or fine curve fitting to the waveform train may be performed as a first step during CDQ assessment. Additionally or alternatively, coarse or fine curve fitting to the waveform train may be performed as a first step during course data sieving. These embodiments may use the fitted curves to perform method steps associated with CDQ or course data sieving. For method steps in which one or more cycles are removed or eliminated by excision, flanking cycles may be stitched together to regenerate a continuous train of cycle waveforms.

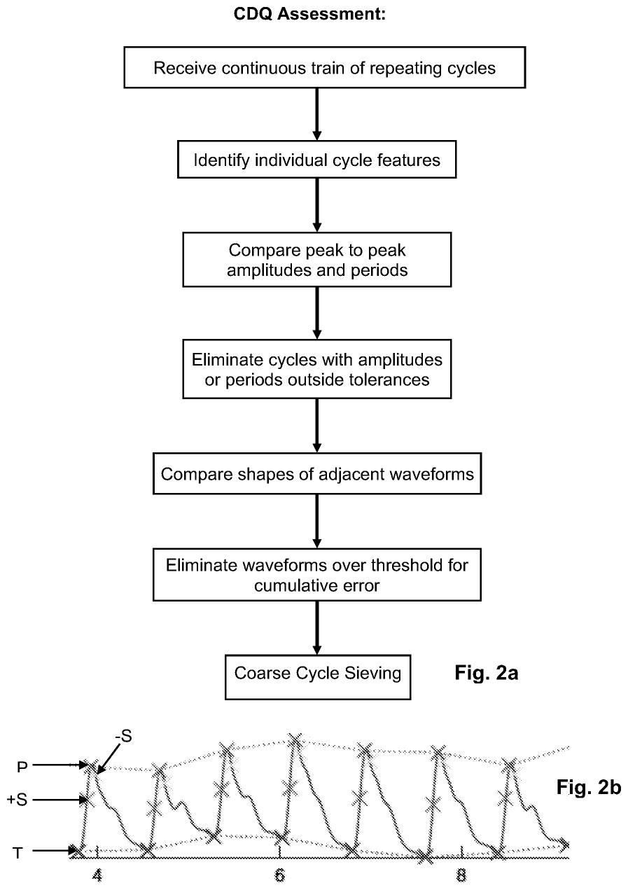

FIG. 2a is a flow chart of method steps that may be performed by one embodiment of a CDQ assessment software module. Data comprising a train of PW cycles from a fingertip PO is received by the module, optionally with data identifying the patient and/or patient parameters such as age, gender, weight, height, and disease state. To prevent artifactual or abnormally shaped PW cycles from leading to failure detection of start, stop, peak, and trough features, or failure of the optimizer, a ratio of standard deviation to mean PPG cycle time may be used to identify high risk cycles before these cycles are processed. If any cycle(s) generates a standard-deviation/mean cycle time ratio greater than a set threshold, the entire cycle(s) may be removed by a process of excision in which the waveform(s) of the cycle(s) are removed along the time axis from the start to the stop or end of the removed cycle(s). The cycles immediately preceding and following the discarded cycle(s) are joined, or stitched, together by shifting the time of the first subsequent remining cycle to match the start time of the preceding cycle that was removed.

PW cycle start, stop, peak, and trough features are detected for each cycle using a slope-threshold crossing technique that guarantees only one slope-crossing feature is detected for each cycle. This allows adjacent cycles to be accurately identified regardless of waveform structure within each cycle. A slope threshold crossing landmark uses PW slope (first derivative of the volume/pressure/absorbance versus time signal) and an exponential-weighted moving average of the absolute value of PW slope to set an adaptive threshold value. The strong positive slope of the systole period generates a characteristic threshold crossing that is independent of cycle vertical drift caused, for example, by subject movement and breathing artifacts, cycle period variability, or variable HR. The slope moving average may be used to derive a second threshold to measure the approximate time of cycle start for each cycle. The PW slope crossing feature is used to initialize and read-out cycle minimum, or trough T and cycle maximum, or peak P values so that minima and maxima are correctly determined as long as the slope-crossing feature detection maintains a one-to-one property with respect to PW cycles.

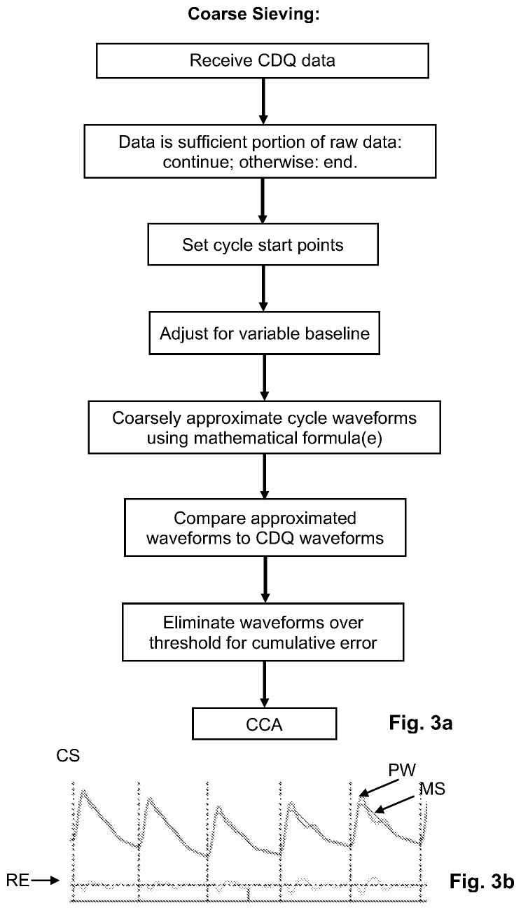

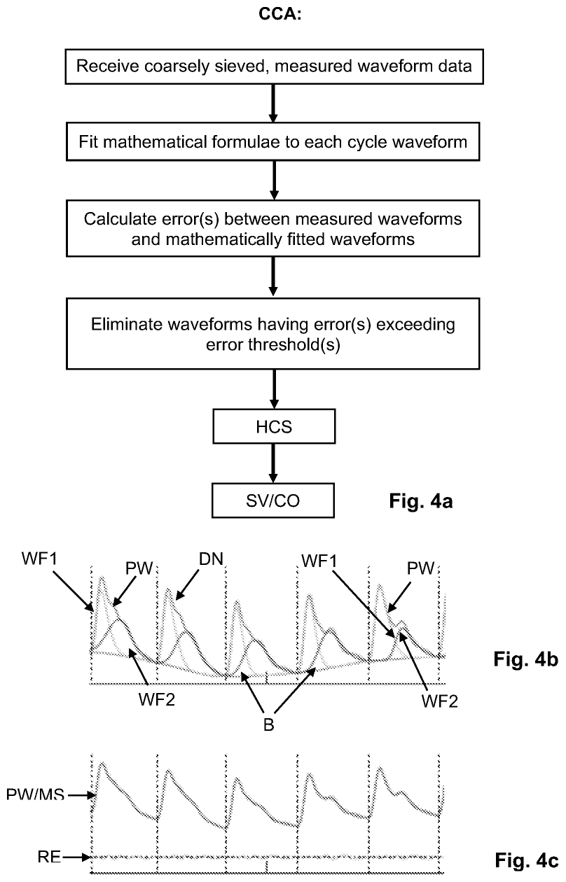

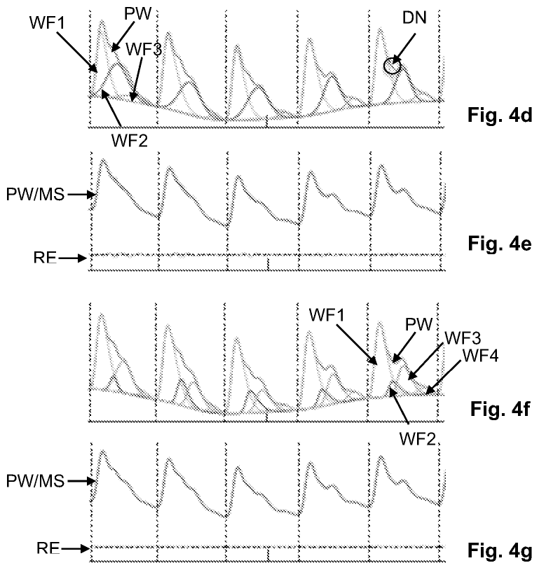

FIG. 2b illustrates that the cycle peak P is found after the maximum positive slope point +S and before the maximum negative slope point −S determined from the first derivative of PW slope. The cycle trough is found after the maximum negative slope point −S and before the maximum positive slope point +S of the next cycle. The top dashed curve in FIG. 2b runs along the cycle peaks P and the bottom dashed curve runs along the cycle troughs T and the amplitude for each cycle is determined from the difference. The bottom dashed curve may serve as a baseline that typically rises and falls in rhythm with the patient's breathing. An “X” is used to mark the positions of each of the troughs T, the maximum positive slope points +S, the cycle peaks P, and maximum negative slope point −S. Any of these positions may be used as the end of one cycle and the beginning of the next. In FIGS. 3b and 4b-4g, the end/start points are marked by vertical dashed lines at the cycle troughs.

The average HR associated with each PW data set is known so there is an expected range of cycle periods associated with the average HR. Once peaks have been identified, peak to peak periods incompatible with the average HR may be eliminated from the data set. Unusual heart rhythms such as premature ventricular contractions (PVCs) and patient movement during data collection can cause cycle peaks to fall outside the tolerance range for an average HR. Similarly, large variations in peak amplitudes are also indicative of unusual heart cycles, patient movements, and other artifacts. Threshold values may be set to remove cycles with amplitudes that differ by more than a tolerated value from adjacent cycles.

The shapes of adjacent cycle waveforms may also be compared to identify cycles that differ significantly from adjacent cycles in terms of waveform shape. The data representing each PW is in the form of volume or pressure vs. time points at a rate, for example, of 75 points per second. The shapes of adjacent cycles may be compared point to point starting with a common landmark for the waves and point to point error may be accumulated to provide a measure of difference between shapes of adjacent cycles. Cycles differing by more than a threshold or tolerance value from neighboring cycles may be removed from the data set before the data is passed to the coarse sieving process. Cycles flanking the removed cycle(s) are stitched together by shifting the start point, along the time axis, of the cycle immediately following the removed cycle(s) to the start time of the first cycle or first of multiple continuous cycles removed.

FIG. 3a is a flow chart of method steps that may be performed by one embodiment of a coarse sieving process. During the CDQ assessment, PWs may be removed for cycles having amplitudes, shapes, periods, etc. that are outside of set tolerance values. In cases where the measured data is of particularly low quality, only a portion of the original data is passed on to the coarse data sieving process. Before the fitting of one or more mathematical formulae in the sieving process, a determination may be made whether a sufficient portion of the original measurement is of sufficient quality for further processing. A threshold value is set below which further processing is halted and an error message generated to inform a user that the measured data is of insufficient quality. For example, for a three minute run of PW measurement, one may set a threshold value of 50% so that, if more than 50% of the cycles are eliminated during the CDQ assessment, a user is notified and the measurement may be repeated. The user may have an option to override the message and proceed with coarse sieving. The threshold value may be set to 100%, effectively eliminating the step and allowing processing to continue, even if all cycles have been eliminated. This functionality may be included as a final step in the CDQ assessment rather than as a first step of coarse sieving.

Once the sufficiency of data is established, cycle start points are assigned to the PW data. The cycle start point CS may be set at the beginning of the systolic wave, or cycle trough, as shown in FIG. 3b. Alternatively, the cycle start CS may be selected in a consistent way anywhere along the cycle waveform. The baseline, which rises and falls with breathing, may be compensated or normalized to provide a flat baseline. Measured PW waveform data often includes lower frequency signal noise from sources including subject breathing and movement effects. This noise, if not subtracted may prevent effective modeling solutions in subsequent steps because skew-Gaussian basis functions alone cannot model a variable baseline. Therefore, this baseline may be modeled using a shape preserving piecewise cubic interpolation provided, for example, by the Matlab® function interp1(‘pchip’). The PW cycle trough points are used as the input to the interpolation. The baseline interpolation output is added to the sum of the skew-Gaussian basis functions in order to model the PW waveform more completely.

A mathematical formula representing one or more waveforms is used to approximate the shapes of the PW cycles. Preferably, one or more skew-Gaussian distribution functions are used but other types of formulae, such as Gaussian and half sine wave distribution functions alone, or in combination, may be used. A least squares solver is initialized with basis function parameter values reasonably close to the global minimum or most desired solution and to set appropriate minimum and maximum boundaries to prevent unwanted local-minimum solutions. An initial, minimum and maximum set of parameters is estimated using the feature detection algorithm in the CDQ assessment module, which is used to locate the start of cycle, systole peak, diastole trough and end of cycle. These feature points may be used in combination with heuristics based on subject population studies to initialize the skew-Gaussian initial, minimum and maximum parameters to optimal values that, through testing and experimentation, have been found to yield stable and accurate pulse PW modeling results. The basis function initial, minimum, and maximum amplitudes are directly calculated from the measured amplitudes of the PW cycles. The basis function initial, minimum, and maximum timing and width (sigma) of the skew-Gaussian waves are based on linear-in-time subdivision of each cycle period. The skewness is preferably initialized to zero.

For each cycle, the shape of the measured cycle is compared to the shape of the mathematically approximated cycle calculate an error value indicating how much the shapes of the measured and approximated cycles differ. Cycles for which the error value is higher than a threshold value are eliminated from the data set. As an example, the difference in shapes may be measured using a least squares analysis using a Levenberg-Marquardt (LM) damped least-squares method. This method is provided with initial, minimum and maximum parameters and an error function. The error function accepts a set of parameters and calculates the sum of the skew-Gaussian basis functions and the interpolated PPG baseline, then subtracts this waveform from the measured PPG waveform to yield an error vector which is returned to the LM solver for analysis and solution iteration.

FIG. 3b shows an example of measured PW cycles and approximated, or modeled, cycles MS superimposed over one another. Accumulated residual error RE for each cycle is shown in the dashed baseline. In this example, the PW cycles are each modeled as a skew-Gaussian distribution function. The RE curve along the baseline can be seen to include noticeable errors. Depending on the threshold value set for acceptable differences, cycles having an error over that value may be eliminated from the cycles forwarded to the CCA module. Significant but subtle differences in shape detected using a coarse, single skew-Gaussian function may provide value in eliminating cycles including noise or artifacts that have not been captured by the CDQ assessment. Vertical dashed lines mark the cycle start CS of each cycle.

FIG. 4a is a flow chart of method steps that may be performed by one embodiment of a cardiac cycle analysis (CCA) process. The CCA algorithm provides a modeling and decomposition, in this example, of fingertip PPG (photoplethysmography) measured cycles (PW cycles) into the sum (superposition) of multiple parameterized skew-Gaussian basis functions. One or more of these basis functions are used to model each cycle. The skew-Gaussian basis functions are defined based on the well-known Gaussian distribution function that is specified with three parameters: amplitude, mean, and sigma or standard deviation. A fourth parameter, the skewness, is incorporated to allow the Gaussian distribution function to exhibit an asymmetry that is measurable and consistent with the statistical measurement of skewness. Each skew-Gaussian basis function is therefore uniquely specified by four parameters: amplitude (PW amplitude units or vertical axis), position (units of time or horizontal axis), sigma (units of time, or width) and skewness (dimensionless). One or more skew-Gaussian basis functions are used to model a single measured PPG (PW) cycle. While the skew-Gaussian basis functions are defined from negative infinity to positive infinity, for efficiency, the extent of these functions is limited in time from slightly before the start of each cycle, through the systole and diastole periods and to beyond the start of the next cycle.

PPG cycles that exhibit reflected waves, including the commonly observed dicrotic notch formation (DN, FIGS. 4b,4d) require two or three skew-Gaussian basis functions and cannot be effectively modeled with only one basis function. PPG cycles that do not exhibit a dicrotic notch or reflected waves can be modeled accurately with only two basis functions and rarely require three or more basis functions. Generally, the first skew-Gaussian basis function (WF1) models well the rise in BP (systole) from the start of a cycle to the BP peak (systolic). The first (WF1) and second (WF2) skew-Gaussian basis functions generally model the diastole decline in BP. The third (WF3, FIGS. 4f,4h) and fourth (WF4, FIG. 4h) skew-Gaussian basis functions are able to model 2nd and 3rd reflected waves, which appear in some subject PPG data. Then the waveforms WF1, WF2, and WF3 in FIG. 4d and the waveforms WF1, WF2, W3 and WF4 in FIG. 4f are combined, the resulting modeled cycles MS are not visually distinguishable from the measured cycles PW in FIGS. 4c and 4g and residual error RE is near zero.

In order to promote maximum cycle modeling accuracy it is preferable that basis functions modeling adjacent cycles are allowed overlap between cycles. For example, a strongly delayed reflected wave may overlap with the next cycle's main wave (or systole). This requires that groups of three cycles must be solved simultaneously to ensure that the center cycle is accurately modeled and that the basis functions that model the center cycle are allowed to extend into the two adjacent cycles. This provides greater accuracy at the expense of additional processing time required.

In order to promote efficient operation and effective testing and verification of the CCA algorithm, it is preferable to operate on segmented time-series data rather than operate on single continuous time series data that could be of unlimited length. Because PPG cycles often span a segment boundary, and in order to provide seamless analysis with no missing cycles and no missing samples, the CCA algorithm processes a current cycle along with preceding and succeeding cycles so that any PPG cycles spanning the cycle edges can be processed as complete cycles. Preceding segments are provided back to the algorithm using a segment-lag scheme to ensure the current segment is complete and seamless and that the preceding segment data can be re-used.

An optimization function such as Isqnonlin or similar program may be used to implement a least squares method, e.g. the Levenberg-Marquardt (LM) damped least-squares method, to solve for an error value for a comparison of the shapes of each of the measured and modeled (approximated) cycles. This method is provided with initial, minimum and maximum parameters and an error function. The error function accepts a set of parameters and calculates the sum of the skew-Gaussian basis functions and the interpolated PPG baseline, then subtracts this waveform from the measured PPG waveform to yield an error vector which is returned to the LM solver for analysis and solution iteration.

Following completion of optimization, the first skew-Gaussian basis function of each PPG cycle can then be calculated independently of other basis functions (reflected waves) and other PPG cycles. This yields a pure skew-Gaussian function that can then be normalized and analyzed to precisely determine the 1% of maximum amplitude point at which the PPG cycle starts. Due to the simplicity and noise-free nature of a single skew-Gaussian function, this point in time can be determined to high precision and sub-sample precision. The precise determination of cycle start for each cycle is important for the accuracy of cardiovascular parameter value calculation by HCS models.

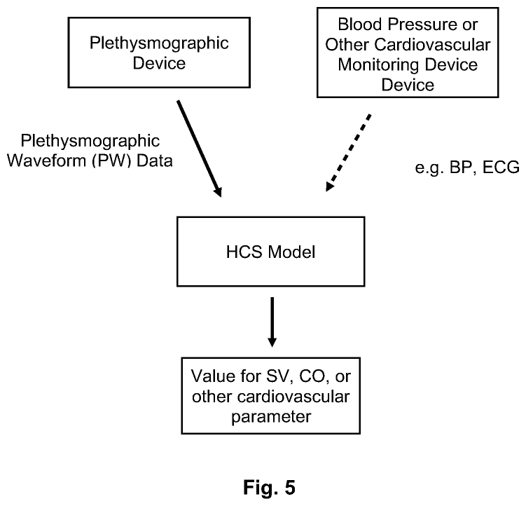

FIG. 5 provides an example of how the cardiac cycle analysis method may be incorporated into a cardiac cycle analysis system. Data from a plethysmographic device, for example a PO, is processed to remove cycles that do not meet set criteria and remaining cycles are used as input for a HCS model (solid arrow). The data may represent measured waveform cycles in the form of data points as measured by the plethysmographic device. Additionally or alternatively, the data may comprise mathematical functions that model the waveforms of measured cycles, also called modeled cycles. This may be particularly useful in cases where high frequency noise is present. Additional data may optionally be made available to the HCS model, for example from a blood pressure monitor or electrocardiogram. The optional additional data may be used by the HCS model as input for cardiovascular parameters.

A system for non-invasively measuring a cardiovascular parameter of a patient may comprise a pulse oximeter, a data collection, storage, and transmission device, a computer, and means for reporting a value for the cardiovascular parameter to a user. The data collection, storage, and transmission device may, for example, a laptop computer or tablet. The pulse oximeter communicates with the data collection, storage, and transmission device and is configured to collect plethysmography waveform data from a patient and transmit the data to the data collection, storage, and transmission device. The data collection and transmission device receives the waveform data and transmits it to the computer. The computer may be the same device as the data collection, storage, and transmission device but the computer may require more computing power and may be a remote cloud computing device. The computer comprises a program of instructions which, when the program is executed by a computer, cause the computer to process the waveform data as described above before providing the resulting output data to a computational model of a cardiovascular system configured to calculate a value for a cardiovascular parameter. The system additionally comprise a blood pressure monitor that sends blood pressure data from the subject to the data collection, storage, and transmission device so that the data can be used as input into the computational model of the cardiovascular system. The data may be collected and transmitted continuously or at intervals such as 1, 2, or 5 minutes. Other noninvasive monitoring devices may also be included to provide input data to the HCS model, such as electrocardiogram data.

The described methods and systems are not limited to physiological systems. For example, they may be applied to other, non-physiological compliant piping systems comprising a positive displacement pump with pulsatile flow and piping system parameters analogous to those of a cardiovascular system in a human or non-human mammal. Although the invention has been described herein with reference to certain preferred embodiments, one skilled in the art will readily appreciate that other applications may be substituted for those set forth herein without departing from the spirit and scope of the present invention.

1. A computer implemented method for processing pulse plethysmography data said data comprising a series of measured cycles, said method comprising:
using a slope-threshold crossing technique to detect a peak, a trough, and a peak amplitude for each measured cycle of the series;
applying a quality metric process to the series of measured cycles to identify measured cycles that do not meet selected quality criteria;
removing the cycles that do not meet selected quality criteria by excision; and
stitching together the measured cycled that have not been removed to provide output data comprising a series of processed measured cycles.

2. The method of claim 1, wherein said applying at least one quality metric process comprises:
comparing a period and amplitude of each measured cycle with a peak amplitude and period of an adjacent measured cycle, or with an average peak amplitude and average period of a plurality of measured cycles and
removing measured cycles having a period or an amplitude outside a tolerance range of variance relative to the peak amplitude and period of the adjacent measured cycle, or the average peak amplitude and average period of the plurality of measured cycles.

3. The method of claim 1, wherein said applying at least one quality metric process comprises:
comparing the shape of each measured cycle with the shape of an adjacent measured cycle, or an average shape of adjacent measured cycles and
removing measured cycles having a shape outside a tolerance range of shape variance with the adjacent measured cycle, or the average of adjacent measured cycles.

4. The method of any of claim 1, wherein said applying at least one quality metric process comprises:
modeling each measured cycle as one or more mathematical functions to provide a series of modeled cycles;
comparing the shape of each measured cycle to the shape of a corresponding modeled cycle to determine a shape variance with the corresponding modeled cycle; and
removing measured cycles having a shape outside a tolerance range of the shape variance with the corresponding modeled cycle.

5. The method of claim 4, wherein said one or more mathematical functions comprises one or more of: a half sine wave function, a square wave function, a Gaussian function, a skew-Gaussian function, a Bessel function, a Fourier series, a Hankel function, a spherical Bessel function, a Struve function, and a Webber function.

6. The method of claim 4, wherein each of said one or more mathematical functions comprises a maximum of ten parameters.

7. The method of claim 5, wherein said one or more mathematical functions comprises at least two skewed Gaussian functions.

8. The method of claim 1, further comprising calculating a value for a cardiovascular parameter wherein said calculating comprises:
providing the output data comprising the series of processed measured cycles to a computational model of a cardiovascular system configured to calculate said value for said cardiovascular parameter; and
causing the computational model to calculate and provide an output reporting said value for said cardiovascular parameter.

9. The method of claim 4, further comprising:
eliminating modeled cycles corresponding to the removed measured cycles to provide a series of processed modeled cycles;
providing the series of processed modeled cycles to a computational model of a cardiovascular system configured to calculate a value for a cardiovascular parameter; and
causing the computational model to calculate and provide an output reporting said value for said cardiovascular parameter.

10. The method of claim 8, wherein the cardiovascular parameter is at least one of stroke volume, cardiac output, aortic pressure, inertance, compliance, resistance, arterial pressure, and venous pressure.

11. The method of claim 8, wherein the computational model of the cardiovascular system is integrated in a dynamic state space model.

12. A system for non-invasively measuring a cardiovascular parameter of a patient, said system comprising:
a pulse oximeter, a data collection, storage, and transmission device, a computer, and means for reporting a value for the cardiovascular parameter to a user wherein:
the pulse oximeter is in communication with the data collection, storage, and transmission device;
the pulse oximeter is configured to collect plethysmography waveform data of the patient, said waveform data comprising a series of measured cycles, and to transmit the plethysmography waveform data to the data collection, storage, and transmission device;
the data collection and transmission device is configured to receive the plethysmography waveform data and transmit the plethysmography waveform to the computer;
the computer comprises a program of instructions which, when the program is executed by a computer, cause the computer to:
use a slope-threshold crossing technique to detect a peak, a trough, and a peak amplitude for each measured cycle of the series of measured cycles;
apply a quality metric process to the series of measured cycles to identify measured cycles that do not meet selected quality criteria;
remove the cycles that do not meet selected quality criteria by excision;
stitch together the measured cycled that have not been removed to provide output data comprising a series of processed measured cycles and/or a series of processed modeled cycles corresponding to the series of processed measured cycles;
provide the output data to a computational model of a cardiovascular system configured to calculate said value for said cardiovascular parameter; and
causing the computational model to calculate and provide an output reporting said value for said cardiovascular parameter.

13. The system of claim 12, further comprising a blood pressure monitor, and wherein:
the blood pressure monitor is in communication with the data collection, storage, and transmission device and is configured to collect blood pressure data of the patient and to transmit the blood pressure data to the data collection, storage, and transmission device and
the computer comprises software that calculates a measured value for the cardiovascular parameter from the blood pressure data and the output data and to report the measured value for the cardiovascular parameter.

14. A computer program product comprising instructions which, when the program is executed by a computer, cause the computer to:
receive pulse plethysmography data comprising a series of measured cycles;
use a slope-threshold crossing technique to detect a peak, a trough, and a peak amplitude for each measured cycle of the series;
apply a quality metric process to the series of measured cycles to identify measured cycles that do not meet selected quality criteria;
removing the cycles that do not meet selected quality criteria by excision;
stitch together the measured cycled that have not been removed to provide output data comprising a series of processed measured cycles and/or a series of processed modeled cycles corresponding to the series of processed measured cycles;
provide the output data to a computational model of a cardiovascular system configured to calculate a value for a cardiovascular parameter; and
calculate and provide an output reporting said value for said cardiovascular parameter.
